(19) United States
(12) Patent Application Publication
(10) Pub. No.: US 2005/0049622 A1
(43) Pub. Date: Mar. 3, 2005

(54) Multi-bladed surgical scalpel

(76) Inventor: Mark Kevin Mittelstaeot, Tucson, AZ (US)

Correspondence Address: MARK MITTELSTAEDT, 5941 E. FORT CRITTENDON TRAIL, TUCSON, AZ, 85750, US

(21) Appl. No.: 10/892,894

(22) Filed: Jul. 16, 2004

Related U.S. Application Data
(63) Continuation-in-part of application No. 09/988,443, filed on Nov. 20, 2001, now abandoned.

(51) Int. Cl. A61B 17/32

(52) U.S. Cl. 606/167

(57) ABSTRACT

This multi-bladed scalpel addresses the problem of making many small incisions in very close proximity to each other, to facilitate hair transplantation. With this device, it is possible to make multiple incisions in such proximity. With the blades mounted parallel to each other, at the desired spacing, each incision does not intersect neighboring incisions, so the follicles placed in each incision will be surrounded by the maximum amount of undisturbed tissue to promote revascularization—capillary growth to provide a blood supply to each transplanted follicle. CNC machining techniques are used to create the blade holders with blade mounting sites created at the desired proximity. Medical grade epoxy is used to mount the blades, which are mounted parallel to each other. Blade mounting holes are drilled so the tips of the blade group form a planar array of interdigitated and offset blades at an angle with respect to the handle.

This specification is a Continuation in Part to application Ser. No. 09/988,443—a multi-bladed surgical scalpel to make incision sites for individual follicle grafts in a surgical process of hair transplantation. The scalpel has an array of interdigitated and irregularly spaced, or offset, blades, such that a line drawn perpendicular to the center of the face of any one blade will not be collinear with a similar line drawn through any other blade. Such scalpel will then are create an array of incisions such that hair follicles can be placed in each incision.

In this embodiment, the blade mounting section of the scalpel is made from an elongate cylindrical section of Delrin; other materials could be used. An array of holes is drilled into the distal circular end face of the Delrin section. The holes are drilled at increasing depth across that end face such that the sharp tips of blades mounted into these holes define a plane. This plane forms a desired angle with respect to the end face of the section. The proximal shafts of the blades are permanently mounted into these holes, such that the blades are oriented parallel to each other. A hole is drilled and tapped along the cylindrical surface of the section of Delrin rod to accommodate mounting and adjustment of the depth gauge.

This same section of Delrin rod has a hole tapped into the proximal face opposite the surface into which the blade shafts are mounted, into which is threaded a setscrew. This setscrew is then used to attach the Delrin section onto the distal tip of an elongate handle, into which the same size setscrew hole has been tapped perpendicular to the end face of said distal tip.

There are external threads on the surface of the distal tip of the handle, immediately below the location onto which the Delrin section containing the blades is mounted. These threads accommodate an adjustment nut, which, when rotated, adjusts the height of the depth gauge.

The depth gauge is machined of elongate tubing of sufficient diameter to fit closely over the Delrin section containing the blades. Said depth gauge has a slot machined into the side of said tubing, such that when the gauge slides over the Delrin section and the slot is located over the tapped hole in the side of the cylindrical surface of the Delrin section, a thumbscrew can be threaded into said tapped hole, to lock the depth gauge in place. The distal tip of the tubing comprising the depth gauge is machined at an angle corresponding to the angle of the blade tips. The depth gauge is adjusted by threading the adjustment nut up or down to expose the desired amount of blade, and thereby determine incision depth. When incision depth is set, the thumbscrew is tightened. Assembly is now complete, and an array of incisions of uniform depth can be made.

### CROSS REFERENCE TO RELATED APPLICATIONS

I found many tools to facilitate the process of making incisions for hair transplants, but none of them could create a planar array of staggered and offset incisions. None had the array of blade tips at some angle with respect to the instrument handle such that when the blades were held at the desired angle to the scalp, all the blades would contact the scalp at the same time. None of them provided the desired blade spacing, angle, or orientation alignment. Typical of these are U.S. Pat. Nos. 5,817,120, 5,782,851, 5,584,841, 5,989,279, 6,022,345, 5,908,417, and 5,733,278, 4,759,363 (Jensen ), U.S. 2001/0034534 A1(Transue), U.S. Pat. No. 5,989,273 (Arnold), etc.

U.S. Pat. No. 5,989,273 is designed to create strips of scalp from which hair follicles to be transplanted are harvested. The blades in U.S. Pat. No. 5,989,273 are secured by a common mounting location and require spacers to determine the distance between slices. While the spacing between the blades could be varied, there is no provision for introduction of blade offset. All the blades must be located along a line. It is not designed to make individual “puncture” incisions, rather, its purpose is to excise strips of donor follicles. Both U.S. Pat. Nos. 5,026,385 and 5,447,516 are similar, and referenced by U.S. Pat. No. 5,989,273. They are designed to take uniform thin slices for the purpose of obtaining strips of donor follicles.

Hair transplantation surgery requires that an array of incisions be created in the scalp of the patient. The incisions need to be offset—not co-linear with each other, and interdigitated with the other incisions, to create a natural appearance. All of the above describe a single, linear arrangement of blades.

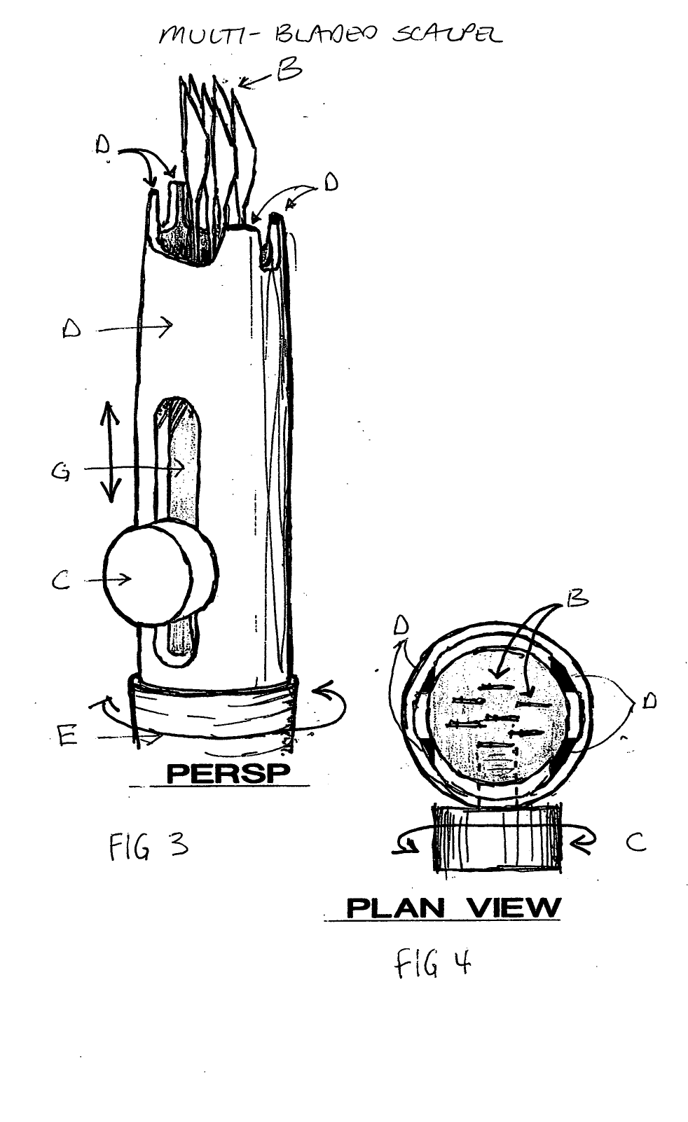

Arnold shows blades at a varying angle to the handle to accommodate the shape of the scalp, in a strictly linear alignment of blades. Arnold provides for staggering of blades (FIG. 13) by loosening a tightening nut. Once staggered at an angle to accommodate the scalp, incisions made by such an apparatus would not be of uniform depth, and therefore unsuitable to make incisions simultaneously, of uniform depth, that could serve as recipient sites for hair follicle grafts.

Transue shows many blades in one handle, and his blades are designed to create recipient sites, but they are “arranged along a single blade axis”. He has no provision for blade stagger, offset, or interdigitation, and is unable to create an array of recipient-site incisions with a single stroke.

Jensen shows a removable, bayonet-style depth gauge, wherein the “blade projects past the tip a predetermined amount”. This design requires removal for readjustment. Also with Jensen, the blade guards “are constructed for right or left hand preference”. The threaded depth gauge described herein allows for continuous, precise incision depth adjustment, and the depth of cut can be seen from all sides, at any angle.

### THIS RESEARCH WAS NOT FUNDED IN ANY WAY BY THE FEDERAL GOVERNMENT. I RETAIN ALL RIGHTS TO THIS INVENTION.

### BACKGROUND—FIELD OF INVENTION

This is a multi-bladed surgical scalpel that makes an array of interdigitated and offset incisions, creating an array of recipient sites for hair follicle transplantation, at uniform depth, in one motion. The incisions are interdigitated and offset such that hair follicles inserted in the incisions will mimic the appearance of a natural hairline.

### OPERATION

The surgeon sets the desired depth of cut by adjusting the depth gauge, then places the scalpel where an array of small incisions is desired. An array of interdigitated recipient-site incisions is made with each stroke of the scalpel.

### INVENTION SUMMARY

Hair transplantation is often performed by a surgical procedure wherein some of a patient's hair follicles are “harvested”, from areas where the patient has hair, by excision of a donor “strip” of hair follicles. This strip is dissected into follicular units, which are then placed in incisions in bald or balding areas of the patient's scalp. This multi-bladed scalpel provides interdigitated and offset incisions that closely match the appearance of natural scalp when follicles are transplanted. With conventional single-bladed knives, a separate stroke is required to make each incision. Extreme care must be taken to locate each incision at the desired location relative to other incisions, make each incision to the desired depth, and align each incision such that it is made at the proper angle into the scalp.

In surgery, this process of making very precise individual incisions is very time consuming. Making the thousands of such precise cuts required during a surgical procedure may result in hand fatigue, and introduces the potential for repetitive stress injury to the surgeon. With multi-bladed scalpels, the amount of time required to make all the necessary incisions is greatly reduced. The percentage of cuts made at the ideal depth, spacing and alignment greatly increases, thus increasing donor hair yield—the number of grafts that survive and grow hair in their transplanted locations.

In one embodiment of this design, an adjustable depth gauge is built into the handle of the knife. This allows the surgeon to set a uniform penetration depth for each array of incisions. Cutting surfaces of the blades are visible from all angles around the depth gauge. Incisions that are too deep bleed more, cause more post-surgical facial swelling, and take longer to heal. It is important to be able to control incision depth precisely. Incisions that are either too deep, or too shallow, may provide undesirable outcomes for a given transplanted hair follicle.

In various embodiments of this invention, different numbers of blades are used, and spacing between the blades is changed to provide varying incision density. This allows the surgeon to more closely match the number of incisions with coverage area desired and amount of donor hair available, as well as make incisions around existing hair follicles.

Also, in various embodiments, the array of blade tips is mounted at varying angles with respect to the handle. This allows the blade tips of a multi-bladed tool to make their individual incisions at the same time when the tool is held at the corresponding angle. Various blade angles provide a tool that can be used at an angle ergonomically suited to the individual surgeon. Incisions are not generally made perpendicular to the scalp, so the blades must be mounted at an angle that corresponds to scalp contours.

These multi-bladed scalpels are inexpensive to manufacture. They require no learning curve to use, or technician to operate. They give the surgeon more time in the time-critical part of the surgery. They decrease hand fatigue, so all incisions can be made more carefully and precisely. The parallel blades provide very repeatable incision spacing and alignment - one incision does not occur too close to another, so capillary revascularization around each follicle is maximized, and post-operative swelling and bleeding are minimized.

### DETAILED DESCRIPTION OF INVENTION

This multi-bladed scalpel is used to create an array of recipient site incisions for hair follicle transplantation. The scalpel creates an array of incisions of uniform depth, then the scalpel is moved to an adjacent area on the scalp to repeat the process. In this embodiment, the blade holder is machined from Delrin, and all the other parts from aluminum, although newer versions might use different materials.

The blade holder is machined from medical grade Delrin, using cylindrical elongate sections. Blade mounting holes are then drilled into the distal end of the section such that the angle of the plane defined by the blade tips is ergonomically desirable angle. The blade holes get deeper as they are drilled from left to right across the distal tip of the blade holder to achieve this angle. A hole is drilled and tapped halfway along the length of the cylindrical face of the section of the rod, and another hole is drilled and tapped on the circular face of the proximal tip of the Delrin section.

The handle in this embodiment is an elongate metal rod, with a hole tapped in the distal tip, and the outside diameter threaded for some distance down from the distal end face containing the tapped hole.

The adjustment nut in this embodiment is machined in the shape of a conic section, with ridges machined along the outside length of the section to provide a non-slip surface. The cone angle is designed to provide an ergonomically correct surface to hold the scalpel. Various embodiments may have varying cone angles and ridge spacings. The inside of the adjustment nut is tapped with threads to provide vertical adjustment of the depth gauge as the adjustment nut is rotated in either direction.

In the assembly process, the cone is threaded down onto the handle, with the smaller diameter end pointing towards the proximal tip of the handle where the blade holder will be attached. The blade holder is then attached.

The distal tip of the depth gauge is machined at an angle that corresponds to the angle of the plane of the blade tips. The top is tapered to minimize wall thickness, and grooved on four sides—left, right, front and back, so blade locations can easily be seen from all sides, leaving four pins to provide depth control. A slot is machined in the side of the depth gauge, such that the slot can be located over the tapped hole in the cylindrical side of the Delrin section when the depth gauge is mounted so the angle of the gauge corresponds to the angle of the blades.

The depth gauge slides over the blade holder, then a thumbscrew is inserted through the slot in the side of the depth gauge into the tapped hole in the side of the Delrin section, and made snug, but not tight. The adjustment nut is advanced or retracted until the desired amount of blade is exposed. Once the precise amount of blade extension is set, the thumbscrew is tightened, and the assembly is ready for sterilization and use.

The blades are mounted parallel to each other, to maximize the amount of undisturbed tissue surrounding each incision.

### DESCRIPTION OF DRAWINGS

Figures one (cross section, front view) and two (cross section, side view) shows one embodiment—a three-bladed scalpel. Many different embodiments are possible, containing varying numbers of blades. The blades, B, are permanently mounted, parallel to each other, to blade holder A, by the use of an attachment means.

The blades are mounted at some angle θ with respect to the top of the handle. An attachment means, in this embodiment thumbscrew C, is used to secure the depth gauge, D, to the handle. Thumbscrew C is inserted into the tapped hole in A by passing it through slot G, as seen in figures one and three

When the adjustment means, in this embodiment an ergonomically-shaped handle grip E with internal threads, is rotated around external threads on handle A, depth gauge D is adjusted vertically until the desired length of blade extends past the tip of the depth gauge D. Thumbscrew C is then tightened to set depth gauge D.

In this embodiment, depth gauge D is tapered at the tip, and channels are cut across the tip, to provide a clear view of the blade location while still providing depth control. Also in this embodiment, the tip angle of depth gauge D is machined such the cut depth is equal for each blade.

Figures three (perspective view) and four (plan view), developed for the continuation-in-part, show another embodiment, this one with seven blades. The planar array of blade tips can be seen more clearly in this embodiment. Also in this embodiment, the depth gauge is shown with material removed from the circumference to improve blade visibility from all angles.

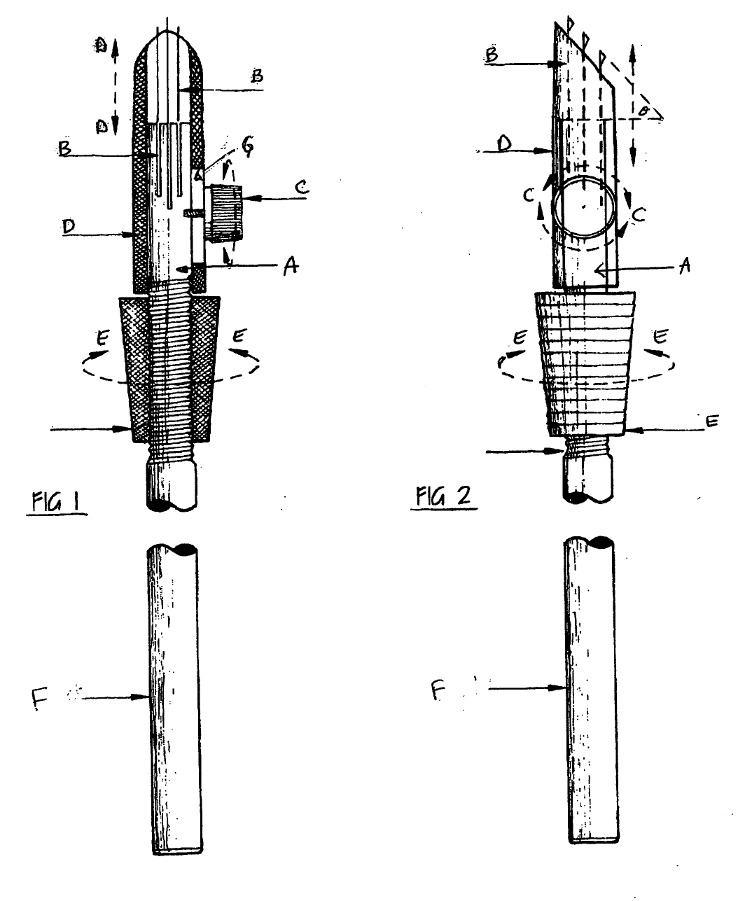

In FIG. 4, the staggered and interdigitated nature of the blades can be more easily seen. The blades do not line up perfectly with each other on any axis.

1.) There is an interdigitated, irregularly-spaced array of multiple scalpel blades at the distal tip of said scalpel.

2.) The interdigitated, irregularly-spaced array of the tips of said blades defines a plane with respect to the distal tip of the scalpel handle.

3.) The depth gauge is continuously adjustable without gauge removal, and blade tip placement can be clearly seen from all angles.

4.) The plane defined by the tips of the depth gauge corresponds to the plane defined by the blade tips, such that the depth of cut of each blade in the array is uniform.
